(19) United States
(12) Patent Application Publication
(10) Pub. No.: US 2021/0040540 A1
(43) Pub. Date: Feb. 11, 2021

(54) PARALLEL LIQUID-PHASE HYBRID CAPTURE METHOD FOR SIMULTANEOUSLY CAPTURING SENSE AND ANTISENSE DOUBLE STRANDS OF GENOMIC TARGET REGION

(71) Applicant: Shanghai Dynastygene Company, Shanghai (CN)

(72) Inventor: Yiqun CHEN, Wuxi (CN)

(21) Appl. No.: 16/619,309

(22) PCT Filed: Apr. 19, 2018
(86) PCT No.: PCT/CN2018/083711

§ 371 Date: Dec. 4, 2019

(51) Int. Cl. C12Q 1/6811; C12N 15/113; C12Q 1/6874

(52) U.S. Cl. CPC C12Q 1/6811; C12Q 1/6874; C12N 15/113

(57) ABSTRACT

The present invention discloses a parallel liquid-phase hybrid capture method of simultaneously capturing sense and antisense double strands of genomic target region. The parallel liquid-phase hybrid capture method disclosed by the present invention comprises: capturing the target DNA using a sense strand probe set and an antisense strand probe set targeting the target DNA to accomplish the capture of the target DNA; the sense strand probe set consists of n sense strand probes, n is greater than or equal to 1; the antisense strand probe set consists of m antisense strand probes, in is greater than or equal to 1; both the sense strand probe set and the antisense strand probe set can cover the entire sequence of the target DNA; each probe in the sense strand probe set and each probe in the antisense strand probe set contain a recognition sequence of a transcriptase and/or a recognition sequence of a sequencing primer. The experiment proves that the present invention can significantly improve the capture efficiency and detection sensitivity of the target DNA in liquid-phase hybrid, and has wide application value in the fields of cancer mutation detection, targeted medication guidance, early screening of fetal genetic defects and infant birth defects, etc.

### RELATED APPLICATIONS

The present application is a National Phase of International Application Number PCT/CN2018/083711, filed Apr. 19, 2018.

### INCORPORATION BY REFERENCE

The sequence listing provided in the file entitled SEQUENCE_LISTING_06-25, which is an ASCII text file that was created on Jun. 25, 2020, and which comprises 61,467 bytes, is hereby incorporated by reference in its entirety.

### TECHNICAL FIELD

The present invention belongs to the field of biotechnology, and particularly relates to the preparation of high-throughput sequencing library, the design of target region probes, the preparation of sense strand probe and antisense strand probe, and the capture and sequencing method of double-strand liquid-phase hybrid enrichment.

### BACKGROUND ART

At present, genetic testing has become an important tool for clinical and scientific research. Next Generation Sequencing (NGS) technology is the most important tool in genetic testing. The high sensitivity and high accuracy of the Next Generation Sequencing technology enable large-scale identification of genetic mutations and sequencing of new species. Although Next Generation Sequencing technology brings greater efficiency to the genome sequencing process, whole genome sequencing still faces the problem that the cost is too high. Therefore, targeted enrichment sequencing of target regions can be rapidly developed. Targeted enrichment sequencing uses various means to capture specific target regions of interest from whole genome libraries for further deep sequencing and analysis. This can greatly reduce the cost of sequencing, and at the same time greatly increase the sequencing depth of the target region, enabling more detailed analysis. Therefore, targeted enrichment sequencing can be more cost-effective for various fields of genetic testing.

At present, large-scale targeted enrichment methods mainly include multiplex PCR method, liquid-phase hybrid method, solid-phase chip capture method, molecular inversion probe method and the like. For most of the larger capture intervals in areas such as cancer and pediatric genetic disease testing, the liquid-phase hybrid method is the most efficient and widely used. However, commercially available liquid-phase hybrid methods at now, regardless of genetic RNA probes or DNA probes, long probes or short probes, only a single strand of each library molecule is captured during hybrid, i.e., only the sense strand or the antisense strand is captured, and then a double-strand library is obtained and complemented by Post-PCR and then analyzed and sequenced. Therefore, no matter how high the capture efficiency, half of the molecules will never be captured, which makes many applications currently severely limited, such as liquid biopsy, the trace samples such as clinical puncture samples, very small amounts of FFPE samples, etc., or low frequency mutations in early detection of cancer, etc. Therefore, how to achieve efficient capture at very low initial amounts, or ultra-low frequency mutation detection under the condition of a common initial amount becomes the most pressing problem.

### SUMMARY OF THE INVENTION

The present invention provides a method of capturing a target DNA, which can simultaneously capture the sense strand and the antisense strand of the target DNA, thereby improving the capture efficiency of the target DNA, and can be used for more efficient detection of low initial amounts or low frequency mutations.

In order to solve the above technical problem, the present invention first provides a method of capturing a target DNA, comprising: capturing the target DNA using a sense strand probe set and an antisense strand probe set targeting the target DNA to accomplish the capture of the target DNA;

the sense strand probe set consists of n sense strand probes, n is greater than or equal to 1; the antisense strand probe set consists of m antisense strand probes, m is greater than or equal to 1.

Each probe of the sense strand probe set binds to one strand of the target DNA; each probe of the antisense strand probe set binds to the other strand of the target DNA.

n can be equal to m.

In the above method, both of the sense strand probe set and the antisense strand probe set can cover the entire sequence of the target DNA.

All of the sense strand probes and the antisense strand probes can be RNA.

When n is greater than 1 or in is greater than 1 and each probe in the sense strand probe set or the antisense strand probes binds to the target DNA, the probes are stacked like tiles, that is, any two adjacent probes on the target DNA satisfy that one or more nucleotides downstream of the upstream probe overlap (are identical to) the upstream of the downstream probe, and when combined with the target DNA, the overlapping portions of the two adjacent probes will bind to the target DNA in the alternative.

In the above methods, each probe in the sense strand probe set and each probe in the antisense strand probe set can contain a recognition sequence of a transcriptase and/or a recognition sequence of a sequencing primer.

In the above method, the transcriptase can be T7 RNA polymerase. The sequencing primer can be P3 and/or P5.

The recognition sequence of the T7 RNA polymerase is positions 1-22 of SEQ ID NO: 1 in the sequence listing. The sequence of P3 can be positions 26-46 of SEQ ID NO: 2 in the sequence listing; the sequence of P5 can be positions 23-41 of SEQ ID NO: 1 in the sequence listing.

In the above methods, the single probe in the sense strand probe set and the antisense strand probe set can have a length of 120-220 nt.

In the above methods, each probe in the sense strand probe set can have a length of 181 nt. Each probe in the antisense strand probe set can have a length of 184 nt.

In the above methods, each probe in the sense strand probe set and the antisense strand probe set can be labeled with biotin.

In the above methods, the capture of the target DNA can be carried out in liquid phase.

In one embodiment of the present invention, the target DNA can be an exon of EGFR, ALK, KRAS and/or BRAF.

The method of preparing the sense strand probes and the antisense strand probes comprises:

1) preparing n original probes according to the target DNA, when each of the original probes is combined with the target DNA, the probes are stacked like tiles, that is, any two adjacent probes on the target DNA satisfy that one or more nucleotides downstream of the upstream probe overlap (are identical to) the upstream of the downstream probe, and when combined with the target DNA, the overlapping portions of the two adjacent probes will bind to the target DNA in the alternative;

2) adding the sequences of two sequencing primers respectively to the two ends of each original probe using PCR to obtain an initial probe, the initial probe being a DNA fragment;

3) adding the recognition sequence of the transcriptase to the 5′ end of one strand of the initial probe by PCR, and then performing reverse transcription to obtain the sense strand probe set;

adding the recognition sequence of the transcriptase to the 5′ end of the other strand of the initial probe by PCR, and then performing reverse transcription to obtain the antisense strand probe set;

the two sequencing primers can be the P5 and the P3, respectively.

Any one of the following uses of the sense strand probe set and the antisense strand probe set is also within the protection scope of the present invention:
X1) use in a target DNA capture;
X2) use in the preparation of a product for a target DNA capture;
X3) use in a target DNA sequencing;
X4) use in the preparation of a product for a target DNA sequencing;
X5) use in genetic disease detection;
X6) use in the preparation of a product for genetic disease detection;
X7) use in cancer detection;
X8) use in the preparation of a product for cancer detection;
X9) use in liquid biopsy;
X10) use in the preparation of a product for liquid biopsy;
X11) use in early screening of a fetal genetic defect;
X12) use in the preparation of a product for early screening of a fetal genetic defect;
X13) use in early screening of an infant birth defect;
X14) use in the preparation of a product for early screening of an infant birth defect;
X15) use in low frequency or ultra-low frequency mutation detection;
X16) use in the preparation of a product for low frequency or ultra-low frequency mutation detection.

Any one of the following uses of the method of capturing a target DNA is also within the protection scope of the present invention:
X1) use in the target DNA capture;
X2) use in the target DNA sequencing;
X3) use in genetic disease detection;
X4) use in cancer detection;
X5) use in liquid biopsy;
X6) use in early screening of a fetal genetic defect;
X7) use in early screening of an infant birth defect;
X8) use in low frequency or ultra-low frequency mutation detection.

### DESCRIPTION OF THE DRAWINGS

FIG. 1 is a flow chart for bidirectional amplification and labeling of the probe set of Example 1. Wherein, PCR1 represents the first step PCR amplification and PCR2 represents the second step PCR amplification.

FIG. 2 is a schematic diagram of bidirectional capture.

FIG. 3 is a comparison of the performance of the sense strand and antisense strand probes parallel liquid-phase hybrid capture and the current standard probe liquid-phase hybrid. Wherein, the unit of the abscissa is %; A is the result of the method of the present invention, and B is the result of the Agilent standard hybrid system.

### DETAILED DESCRIPTION OF THE INVENTION

The present invention is further described in detail below with reference to the specific embodiments, and the examples are given only to illustrate the present invention and not to limit the scope of the present invention. The experimental methods in the following examples are conventional methods unless otherwise specified. The materials, reagents, instruments and the like used in the following examples are all commercially available unless otherwise specified. For the quantitative tests in the following examples, they are repeated three times, and the results are averaged. In the following examples, unless otherwise specified, the first position of each nucleotide sequence in the sequence listing is the 5′ end nucleotide of the corresponding DNA, and the last position is the 3′ end nucleotide of the corresponding DNA.

### EXAMPLE 1

### Preparation of Liquid-Phase Capture Initial Probe Set

1. According to the sequences of the exons of the target genes (EGFR, ALK, KRAS, BRAF), four sets of original probes stacked like tiles were designed. Each of the original probes in the four sets of probes was 120 mer in length and was single-stranded DNA, each set of probes could cover the entire sequence of the exon of the target gene, and when it bound to the exon of the target gene, any two adjacent original probes could satisfy that 60 bp nucleotides downstream of the upstream original probe overlapped the upstream of the downstream original probe (i.e., the 60 by nucleotides downstream of the upstream original probe were identical to the upstream sequence of the downstream original probe). The sequences of the probes of ALK are SEQ ID NOs: 1-99 in the sequence listing, the sequences of the probes of EGFR are SEQ ID NOs: 100-182 in the sequence listing, the sequences of the probes of KRAS are SEQ NOs: 183-234 in the sequence listing, the sequences of the probes of BRAF are SEQ ID NOs: 235-248 in the sequence listing.

2. The sequence of 5′-ATGCGACGTCGCAGT-3′ was added to the 5′ end of each original probe obtained in step 1, and the sequence of 5′-CTGCCTGGTCCGACA-3′ was added to the 3′ end. The total length of each probe was 150 bp. The obtained single probe was named as an initial probe, and the probe set composed of each initial probe was named as an initial probe set, i.e., a liquid-phase capture initial probe set.

3. All the initial probes obtained in step 2 were pooled (probe pool synthesis was carried out on OligoArrav instrument purchased from CustomArray). The mole number of each probe in the initial probe set was equal.

### EXAMPLE 2

### Bidirectional Amplification and Labeling of the Probe Set of Example 1

The probe set of Example 1 was bidirectionally amplified and labeled, and the process is shown in FIG. 1. The Herculase kit used is Agilent's product and its catalog number is 600677. The specific method was as follows:

### 1. The Initial Probe Set Obtained in Example 1 was Diluted to 152.8 μL to Obtain an Initial Probe Solution, and the First Step PCR Amplification Was Carried Out, and the Reaction System (Total Volume: 200 μL) of the Amplification was as Follows:

| | | |
|---|---|---|
| 1.6 | μL | P5 primer (10 μM) (sequence: |
| | | AGGGATGCGACGTCGCAGT); |
| 1.6 | μL | P3 primer (10 μM) (sequence: |
| | | GTGGACTGCCTGGTCCGACA); |
| 2 | μL | dNTP mixture (each dNTP |
| | | concentration: 100 mM) |
| | | (a reagent in the Herculase kit); |
| 40 | μL | 5× Herculase II Reaction Buffer |
| | | (a reagent in the Herculase kit); |
| 2 | μL | Herculase II Fusion DNA Polymerase |
| | | (a reagent in the Herculase kit); |
| 152.8 | μL | the initial probe solution. |

The above system was evenly mixed and divided into four PCR tubes, 50 μL per tube. The reaction was carried out according to the following reaction conditions:

| | | | | |
|---|---|---|---|---|
| 98° C. | 2 | min | | |
| 98° C. | 30 | s | | |
| 55° C. | 30 | s | {close oversize brace} | 10 cycles |
| 72° C. | 30 | s | | |
| 72° C. | 10 | min | | |

| | |
|---|---|
| 4° C. | hold |

The reaction products obtained in the four PCR tubes were combined, and then 360 μL of AMpure magnetic beads were added thereto for purification, and rinsed with 500 μL of ethanol. Finally, the product was eluted with deionized water to obtain 152.8 μL of the first step amplification probe solution.

### 2. The Second Step PCR Amplification of the Probe Set:

The reaction system for preparing the sense strand probe (total volume: 200 μL) was as follows:
1.6 μL P5-T7 primer (10 μM);
1.6 μL P3 primer (10 μM);
2 μL dNTP mixture (each dNTP concentration: 100 mM) (a reagent in the Herculase kit);
40 μL 5×Herculase II Reaction Buffer (a reagent in the Herculase kit);
2 μL Herculase II Fusion DNA Polymerase (a reagent in the Herculase kit);
152.8 μL the first step amplification probe solution.

Wherein, the sequence of the P5-T7 primer is

(SEQ ID NO: 249
GGATTCTAATACGACTCACTATAGGGATGCGACGTCGCACT

in the sequence listing).

The reaction system for preparing the antisense strand probe (total volume: 200 μL) was as follows:
1.6 μL P5 primer (10 μM);
1.6 μL P3-T7 primer (10 μM);
2 μL dNTP mixture (each dNTP concentration: 100 mM) (a reagent in the Herculase kit);
40 μL 5×Herculase II Reaction Buffer (a reagent in the Herculase kit);
2 μL Herculase II Fusion DNA Polymerase (a reagent in the Herculase kit);
152.8 μL the first step amplification probe solution.

Wherein, the sequence of the P3-T7 primer is

(SEQ ID NO: 250
GGATTCTAATACGACTCACTATAGGGTGGACTGCCTGGTCCGACA

in the sequence listing).

For the above two reaction systems, each reaction system was evenly mixed and divided into four PCR tubes, 50 μL per tube. The reaction was carried out according to the following reaction conditions:

| | | | | |
|---|---|---|---|---|
| 98° C. | 2 | min | | |
| 98° C. | 30 | s | | |
| 55° C. | 30 | s | {close oversize brace} | 10 cycles |
| 72° C. | 30 | s | | |
| 72° C. | 10 | min | | |

| | |
|---|---|
| 4° C. | hold |

After the reaction was completed, the four PCR tubes containing the sense strand probe PCR reaction products and the four PCR tubes containing the antisense strand probe PCR reaction products were obtained. The four PCR tubes containing the sense strand probe PCR reaction products and the four PCR tubes containing the antisense strand probe PCR reaction products were separately combined, and then the two probes were purified according to the following steps: 360 μL of AMpure magnetic beads (Beckman, catalog number: A63880) was added to the probe after the combination, evenly mixed and allowed to stand at room temperature for 5 min, placed on a magnetic stand for 2 min to be clarified, and the supernatant was aspirated. 500 μL of ethanol was added to the magnetic beads, evenly mixed and allowed to stand for 2 min. The ethanol was removed completely after the solution was clarified, and allowed to dry at room temperature for 5 min. Finally, 25 μL of nuclease-free water was added to resuspend the magnetic beads. After standing for 2 min, the supernatant was aspirated and transferred to a new tube to obtain the target probe.

### 3. The Labeling of the Probes

The sense strand probe PCR reaction product and the antisense strand probe PCR reaction product obtained in step 2 were subjected to in vitro transcription and biotin labeling as follows:

1 μg of the PCR reaction product was taken, adjusted to a final volume of 28.2 μL with nuclease-free water, and then the following reagents were added thereto to obtain an in vitro transcription and labeling system (total volume: 50 μL):
5 μL 10×Transcription Buffer (consisting of a solvent and solutes, the solvent was 0.4 M Tris-HCl (pH 8.0), the solutes and the concentrations thereof were 60 mM MgCl₂, 100 mM DTT (dithiothreitol), 20 mM spermidine);
12.5 μL Biotin RNA Labeling Mix (Biotin Labeling Mix, Roche, catalog number: 11685597910);
1.5 μL T7 RNA Polymerase (20 U/ml, Roche);
0.3 μL Pyrophosphatase (0.1 U/ml, NEB);
2.5 μL SUPERase-In RNase inhibitor (20 U/ml, Life Technologies, RNase inhibitor).

The labeling system was evenly mixed by gently pipetting up and down and incubated at 37° C. for 16 h to obtain a labeled transcription reaction product;

1 μL of TURBO DNase (2 U/μL, Ambion) was added to the labeled transcription reaction product to digest for 15 min at 37° C., then purified with Qiagen's RNeasy Mini Kit, eluted with 50 μL of nuclease-free water twice. Two elution products were combined to obtain 100 μL of labeled probe solution.

The final RNA yield was measured using NanoDrop's RNA-40 and Qubit RNA Kit, and the probe distribution was detected using a 2100 RNA Kit, and the size of the obtained RNA was confirmed to be about 150 nt.

The concentration of the probe in the labeled probe solution was adjusted to 200 ng/μL with nuclease-free water, and the SUPERase-in RNase inhibitor (Ambion) was added thereto at a final concentration of 1 U/ul of the probe, and stored at −80° C. Finally, a biotin-labeled sense strand capture probe solution and a biotin-labeled antisense strand capture probe solution were obtained.

### EXAMPLE 3

### Parallel Liquid-Phase Hybrid Capture and Sequencing of the Sense and Antisense Strand Probes

1. 30 ng of plasma free DNA standard (Horizon, catalog number: HD780) was taken for constructing a library, and a genomic DNA library was obtained. The kit used for constructing the library is (Wuxi Diying Biotechnology Co., Ltd., catalog number: D8010A), and the method was as follows:

Water was added to 30 ng of plasma free DNA standard to a final volume of 54.7 μL, then 9.8 μL of end-repair buffer (blue cap, No. 1 tube) and 5.5 μL of end-repairase (blue cap, No. 2 tube) were added, evenly mixed by pipetting up and down, placed on a PCR instrument and incubated at 20° C. for 30 min. The hot lid was not applicable. 120 μL of AMPure magnetic beads (ensured to be equilibrated for more than 30 min at room temperature) was added and evenly mixed. The resulting mixture was allowed to stand at room temperature for 5 min, taking care not to place it on a magnetic stand during this period. Then the mixture was placed on a magnetic stand and allowed to stand for clarification and the supernatant was discarded. 200 μL of 80% ethanol (formulated on the same day) was added, allowed to stand it for 1 min, and the supernatant was discarded. 200 μL of 80% ethanol (formulated on the same day) was added again, and after standing for 30 s, the supernatant was discarded, and the residual ethanol was discarded after rapid centrifugation and the tube was allowed to stand at room temperature for 3 min. The tube was removed from the magnetic stand, 42 μL of nuclease-free water was added to resuspend the magnetic beads, then 6 μL of tailing buffer (green cap, No. 3 tube) and 2 μL of tailing enzyme (green cap, No. 4 tube) were added and well mixed with a vortex mixer, incubated at 30° C. for 30 min without heating the lid. 90 μL of purification and binding solution (yellow cap, No. 5 tube, equilibrated for 30 min at room temperature) was added and well mixed. The tube was removed from the magnetic stand, 30 μL of nuclease-free water was added to resuspend the magnetic beads, and 15 μL of ligation buffer (orange cap, No. 6 tube) and 5 μL of the adapter mix (brown cap, No. 7 tube) was then added. The mixture was well mixed with a vortex mixer. The tube was placed on a PCR instrument and incubated at 20° C. for 15 min, without heating the lid. 70 μL of purification and binding solution (yellow cap, No. 5 tube) was added, the DNA was purified and finally eluted with 20 μL of nuclease-free water. 25 μL PCR mix (pink lid, No. 8 tube), 5 μL of Pre-PCR primer mix (white cap, No. 9 tube) and 20 μL of eluted DNA were added to a new PCR tube. The mixture was well mixed. PCR amplification was performed according to the following PCR conditions.

### Step Temperature Time

| | | | |
|---|---|---|---|
| S1 | 98° C. | 45 | s |
| S2 | 98° C. | 15 | s |
| S3 | 65° C. | 30 | s |
| S4 | 72° C. | 30 | s |

| | |
|---|---|
| S5 | repeal S2-S4 6-8 times |

| | | | |
|---|---|---|---|
| S6 | 72° C. | 5 | min |

| | | |
|---|---|---|
| S7 | 4° C. | hold |

70 μL of AMPure magnetic beads were added to the PCR tube and finally eluted with 30 μL of nuclease-free water.

750 ng of genomic DNA library was taken and water was added to a final volume of 50 μL. After purified with 1.8× magnetic beads, the mixture was eluted with the following reagents. First, 6.4 μL of nuclease-free water was added, then 2.5 μL of human Cot-1 DNA (Thermo Fisher, catalog number: 15279911, 1 mg/ml), 2.5 μL of salmon sperm DNA (Salmon sperm DNA, Thermo Fisher, catalog number: 15632-011, 10 mg/ml) and 0.6 μL of Blocker 3 (Wuxi Diying Biotechnology Co., Ltd., catalog number: D8014A) were added.

2. 11-12 μL of the eluted product obtained in step 1 was placed on a PCR instrument, incubated at 95° C. for 5 min, and maintained at 65° C. for at least 5 min to obtain a reaction product;

3. The following mixture was prepared according to the sample amount in a 1.5 mL EP tube: 6.63 μL of 20× SSPE, 0.27 μL of 0.5M EDTA, 2.65 μL of 50×Denhardt's (Thermo Fisher, catalog number: 750018), 3.45 μL of 1% SDS were sequentially added to the EP tube. The mixture was vortexed vigorously for 2 s and then spined (centrifuged with a high speed). If there was sediment, the tube was incubated at 65° C. for 5 min; then 1 μL of SUPERase-In RNase inhibitor (20 U/μL) and 1 μL of the sense strand capture probe solution and 1 μL of the antisense strand capture probe solution of Example 2 were added to the EP tube, the mixture was vortexed vigorously for 5 s and then spined, and well mixed to obtain a mixed solution.

4. 16 μL of the solution of the above step 3 was added to the reaction product of step 2, and gently pipetted up and down 10 times to obtain a hybrid system.

5. After step 4 was completed, the hybrid system was sealed with a lid and hybridization was conducted at 65° C. for 16 h to obtain a hybrid product.

6. 50 μL of Dynabeads MyOne Streptavidin T1 beads (Life Technologies) was prepared, eluted with Bead binding solution, 200 μL of the obtained magnetic bead suspension was added to the hybrid product of step 5, and incubated for 30 min at room temperature on a rotary mixer. Then, the mixture was adsorbed on a magnetic stand, and the supernatant was discarded, 200 μL of high-salt eluent (Wuxi Diving Biotechnology Co., Ltd., D8013A) was added and incubated for 15 min at room temperature. The mixture was adsorbed on the magnetic stand and the supernatant was discarded. Then low salt eluent (Wuxi Diying Biotechnology Co., Ltd., D8013A) preheated at 65° C. was added and the mixture was washed a total of three times. Finally, 31.5 μL of water was added to the magnetic beads to obtain a purified product.

7. 10 μL of 5×Herculase II Reaction Buffer, 1 μL of Herculase II Fusion DNA Polymerase, 0.5 μL of dNTP mixture (each dNTP concentration: 100 mM), 1 μL of 12.5×SYBR Green and 1 μL of DYPostPCR-U were sequentially added to the purified product obtained in step 6. Then 5 μL of different DYPostPCR-I was added to each sample to obtain different amplification systems,

The 5×Herculase II Reaction Buffer, Herculase II Fusion DNA Polymerase and dNTP mixture all are reagents in the Herculase kit.

The amplification primer sequences are as follows:

DYPostPCR-U:
5′-
AATGATACGGCGACCACCGAGATCTACACTCTTTCCCTACACGACG
CTCTTCCGATC*T-3′;
DYPostPCR-I:
5′-CAAGCAGAAGACGGCATACGAGATYYYYYYYYGTGACTGGAGT
T*C-3′.

YYYYYYYY represents an index sequence of 8 bp in size for distinguishing the samples, such as GCCACATA, CTGGCATA, etc., and the sequence only needs to ensure that the DYPostPCR-I are different in different samples.

The symbol * in the DYPostPCR-U and DYPostPCR-I sequences indicates a thio modification.

Different amplification systems were reacted under the following conditions to obtain amplification products: 98° C. for 2 min; 98° C. or 30 s, 57° C. for 30 s, 72° C. for 60 s, 16 cycles; 72° C. for 10 min.

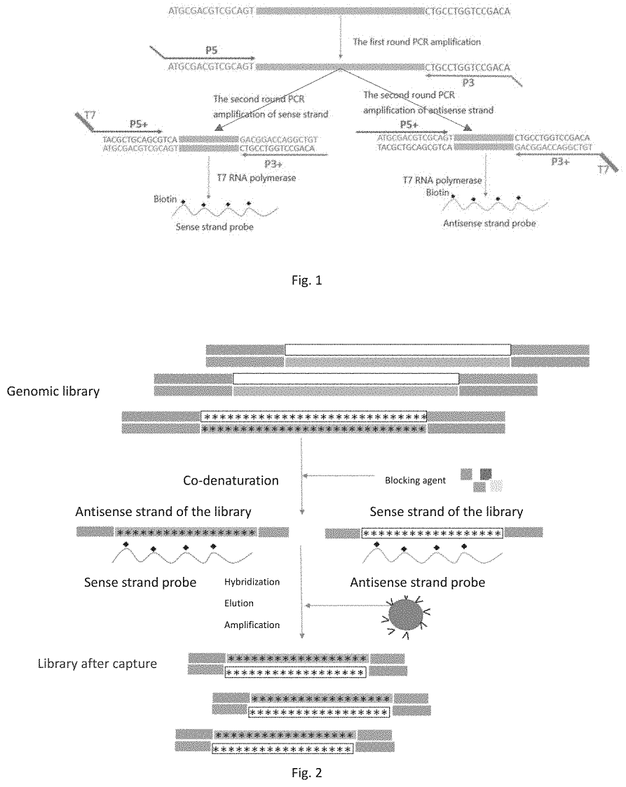

8. After step 7 was completed, 40 μL of water was added to the obtained amplification product, and then 90 μL of AMPure magnetic beads were added, the mixture was purified and eluted with water to 20 μL to obtain a purified product, that is, a library after capture, and the preparation process of the library after capture is shown in FIG. 2.

9. The purified product obtained in step 8 was sequenced using Illumina's HiSeq NGS platform, using a 2×150 bp paired-end sequencing mode. The amount of data required for sequencing was calculated based on the size of the Panel (the entire area in which the probe was designed).

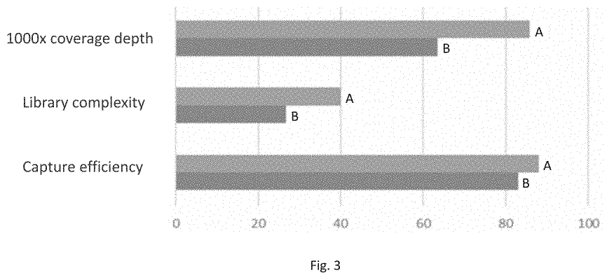

The result is shown in FIG. 3. It can be seen from the figure that, compared to the industry-recognized Agilent standard hybrid system (Agilent G9611A), the capture efficiency of capturing the target DNA using the sense strand capture probe and the antisense strand capture probe simultaneously, the library complexity (ratio of unique Reads in the library at a particular sequencing depth) and the greater than 1000× coverage of the sequencing of the captured DNA of the present invention all were significantly greater than the Agilent standard hybrid system.

### INDUSTRIAL APPLICATION

The experiment proves that the method of capturing a target DNA of the present invention can significantly improve the capture efficiency and detection sensitivity of the DNA in liquid-phase hybrid. A significant increase in library complexity after capture can ensure that more original variation information can be obtained as much as possible under the condition that the initial amount is ensured, and is especially suitable for the samples with low initial amount and low frequency mutation detection. The method of capturing a target DNA of the present invention has wide application value in the fields of cancer mutation detection, targeted medication guidance, fetal genetic defects and early screening of birth defects of infants, etc.

What is claimed is:

1. A method of capturing a target DNA, comprising: capturing the target DNA using a sense strand probe set and an antisense strand probe set targeting the target DNA to accomplish the capture of the target DNA;
the sense strand probe set consists of n sense strand probes, n is greater than or equal to 1;
the antisense strand probe set consists of m antisense strand probes, m is greater than or equal to 1.

2. The method according to claim 1, wherein both of the sense strand probe set and the antisense strand probe set can cover the entire sequence of the target DNA.

3. The method according to claim 1, wherein each probe in the sense strand probe set and each probe in the antisense strand probe set further contain a recognition sequence of a transcriptase and/or a recognition sequence of a sequencing primer.

4. The method according to claim 3, wherein the transcriptase is T7 RNA polymerase; the sequencing primer is P3 and/or P5.

5-10. (canceled)

11. The method according to claim 3, wherein the method of preparing the sense strand probes and the antisense strand probes comprises:
1) preparing n original probes according to the target DNA, when each of the original probes is combined with the target DNA, the probes are stacked like tiles, that is, any two adjacent probes on the target DNA satisfy that one or more nucleotides downstream of the upstream probe overlap (are identical to) the upstream of the downstream probe, and when combined with the target DNA, the overlapping portions of the two adjacent probes will bind to the target DNA in the alternative;
2) adding the sequences of two sequencing primers respectively to the two ends of each original probe using PCR to obtain an initial probe, the initial probe being a DNA fragment;
3) adding the recognition sequence of the transcriptase to the 5′ end of one strand of the initial probe by PCR, and then performing reverse transcription to obtain the sense strand probe set.

12. The method according to claim 1, wherein the single probe in the sense strand probe set and the antisense strand probe set has a length of 120-220 nt.

13. The method according to claim 1, wherein each probe in the sense strand probe set has a length of 181 nt; and/or each probe in the antisense strand probe set has a length of 184 nt.

14. The method according to claim 1, wherein each of the sense strand probe set and the antisense strand probe set is labeled with biotin.

15. A method of sequencing target DNA, comprising the method of capturing a target DNA according to claim 1.

16. A method of cancer detection, comprising the method of capturing a target DNA according to claim 1.


SEQUENCE LISTING (250 sequences)

SEQ ID NO: 1 - DNA, 120 bp, Artificial Sequence
  Synthesized
tgcgaccgagctcagggcccaggctggttcatgctattcttgcttttcagaatggtatcctcgtaatgaccagctccaggggcagtagcggcttctaagggcaagccctgttgctggtag

SEQ ID NO: 2 - DNA, 120 bp, Artificial Sequence
  Synthesized
tcgtaatgaccagctccaggggcagtagcggcttctaagggcaagccctgttgctggtagccgtaattgacattcccacaagggaagtgacgtagcctgaacagaggtacctccttcata

SEQ ID NO: 3 - DNA, 120 bp, Artificial Sequence
  Synthesized
ccgtaattgacattcccacaagggaagtgacgtagcctgaacagaggtacctccttcatattggcagtcagcgaagagggctctaggagcagtgaggcccccggaagtctcccagttgca

SEQ ID NO: 4 - DNA, 120 bp, Artificial Sequence
  Synthesized
ttggcagtcagcgaagagggctctaggagcagtgaggcccccggaagtctcccagttgcaacgttaggtgggacagtacagcttccctccagccccaggttacccctgtcgtgtggctcc

SEQ ID NO: 5 - DNA, 120 bp, Artificial Sequence
  Synthesized
acgttaggtgggacagtacagcttccctccagccccaggttacccctgtcgtgtggctccttctttgctataggattattctttttggtgggtttctctgtaaaccaggagccgtacgtt

SEQ ID NO: 6 - DNA, 120 bp, Artificial Sequence
  Synthesized
ttctttgctataggattattctttttggtgggtttctctgtaaaccaggagccgtacgttgggttccacaagctggtgggcttgtttctggatccgtggaccttgtgcaactccgaagga

SEQ ID NO: 7 - DNA, 120 bp, Artificial Sequence
  Synthesized
gggttccacaagctggtgggcttgtttctggatccgtggaccttgtgcaactccgaaggagggttggactgagagaatgccatattcacgtgtcccccttccacggccggccctctaggg

SEQ ID NO: 8 - DNA, 120 bp, Artificial Sequence
  Synthesized
gggttggactgagagaatgccatattcacgtgtcccccttccacggccggccctctagggactcgaacagagatctctgcagctgtgggtttctttgcagccttgccagaggaggtggta

SEQ ID NO: 9 - DNA, 120 bp, Artificial Sequence
  Synthesized
actcgaacagagatctctgcagctgtgggtttctttgcagccttgccagaggaggtggtaggcagaggtggtggggcagctgggctgcgctcctcctcccgttttgcctgttgagagacc

SEQ ID NO: 10 - DNA, 120 bp, Artificial Sequence
  Synthesized
ggcagaggtggtggggcagctgggctgcgctcctcctcccgttttgcctgttgagagaccaggagaggaggaaccccctcagggtccttgggcctcacaggcactttctcttcctcttcc

SEQ ID NO: 11 - DNA, 120 bp, Artificial Sequence
  Synthesized
aggagaggaggaaccccctcagggtccttgggcctcacaggcactttctcttcctcttccacaagtggaccatattctatcggcaaagcggtgttgattacatccgggtcctgccgtagg

SEQ ID NO: 12 - DNA, 120 bp, Artificial Sequence
  Synthesized
gtttcatatagagtaaatgttgaccaaagggagaaaatgttttacctgggtgcagtattcaatcctctccaaaatgatggcaaagttgggcctgtcttcaggctgatgttgccagcactg

SEQ ID NO: 13 - DNA, 120 bp, Artificial Sequence
  Synthesized
aatcctctccaaaatgatggcaaagttgggcctgtcttcaggctgatgttgccagcactgagtcattatccggtatctaaaagaagaagcacattaattaaaataaggagaagcacaatg

SEQ ID NO: 14 - DNA, 120 bp, Artificial Sequence
  Synthesized
tgaaaagaaaaactgcttagtaactagcagaagtgttcctaaaagagtcatacacaggcccagggcagttcttgggtgggtccatccggcctccactggtgacaaactccagaacttcct

SEQ ID NO: 15 - DNA, 120 bp, Artificial Sequence
  Synthesized
cagggcagttcttgggtgggtccatccggcctccactggtgacaaactccagaacttcctggttgcttttgctggggtatggcatatatccaagagaaaagatttcccatagcagcactc

SEQ ID NO: 16 - DNA, 120 bp, Artificial Sequence
  Synthesized
ggttgcttttgctggggtatggcatatatccaagagaaaagatttcccatagcagcactccaaaggacctgggcatgggacagaggacatggagatggatatagacacacccacccacat

SEQ ID NO: 17 - DNA, 120 bp, Artificial Sequence
  Synthesized
atggctgcagggatacctggaggatgatggctgacttaccatgtgtctgttttagaagtgaatattccttccatgaaggcctctgggggcatccacttaactggcagcatggcacagcct

SEQ ID NO: 18 - DNA, 120 bp, Artificial Sequence
  Synthesized
aatattccttccatgaaggcctctgggggcatccacttaactggcagcatggcacagcctccctttctatagtagctcgccctgtggggaaggagaggaaaaccaaactaggatctggag

SEQ ID NO: 19 - DNA, 120 bp, Artificial Sequence
  Synthesized
tgtgttacaggaagcagcttcctgccagctgtcgacattaattaattacctcaataaataagtactgaatatttagtatgtatcaggtactctgccatgtgctgaggacataaataggtc

SEQ ID NO: 20 - DNA, 120 bp, Artificial Sequence
  Synthesized
agtactgaatatttagtatgtatcaggtactctgccatgtgctgaggacataaataggtcagtctctctctcccaaggatattgtttagtggaagaaatggatatatatgcattgcaata

SEQ ID NO: 21 - DNA, 120 bp, Artificial Sequence
  Synthesized
ggtggaagagacaggcccggaggggtgaggcagtctttactcacctgtagatgtctcgggccatcccgaagtctccaatcttggccactcttccagggcctggacaggtcaagaggcagt

SEQ ID NO: 22 - DNA, 120 bp, Artificial Sequence
  Synthesized
ccatcccgaagtctccaatcttggccactcttccagggcctggacaggtcaagaggcagtttctggcagcaatgtctctgggaagaaaggaaatgcatttcctaattttatccctaggaa

SEQ ID NO: 23 - DNA, 120 bp, Artificial Sequence
  Synthesized
agcgacaggatgacaggaagagcacagtcactttgactcaccggtggatgaagtggttttcctccaaatactgacagccacaggcaatgtcccgagccacgtgcagaaggtccagcatgg

SEQ ID NO: 24 - DNA, 120 bp, Artificial Sequence
  Synthesized
cctccaaatactgacagccacaggcaatgtcccgagccacgtgcagaaggtccagcatggccagggaggagggctggctctgtggggagacagaagcgggccactgacgaggagcttgtc

SEQ ID NO: 25 - DNA, 120 bp, Artificial Sequence
  Synthesized
atccttgctcctgtccttggcacaacaactgcagcaaagactggttctcactcaccgggcgagggcgggtctctcggaggaaggacttgaggtctccccccgccatgagctccagcagga

SEQ ID NO: 26 - DNA, 120 bp, Artificial Sequence
  Synthesized
gagggcgggtctctcggaggaaggacttgaggtctccccccgccatgagctccagcaggatgaaccggggcagggattgcaggctcaccccaatgcagcgaacaatgttctggtggttga

SEQ ID NO: 27 - DNA, 120 bp, Artificial Sequence
  Synthesized
tgaaccggggcagggattgcaggctcaccccaatgcagcgaacaatgttctggtggttgaatttgctgcagagcagagagggatgtaaccaaaattaactgagctgagtctgggcaaatc

SEQ ID NO: 28 - DNA, 120 bp, Artificial Sequence
  Synthesized
gggcagcaggggtcccgggctgagcctaaacccagaatcttgctggtgagcaggtgggaagaaccacagcaggctccacaggagttccatttgcaggagagtggctggagctgtgaggat

SEQ ID NO: 29 - DNA, 120 bp, Artificial Sequence
  Synthesized
gaaccacagcaggctccacaggagttccatttgcaggagagtggctggagctgtgaggatgttgctcaggcacttgggtgaggaagtgtctcagggggcaggagagtgtctttctcagat

SEQ ID NO: 30 - DNA, 120 bp, Artificial Sequence
  Synthesized
gttgctcaggcacttgggtgaggaagtgtctcagggggcaggagagtgtctttctcagatactggtgcgtggactgggtgctagaattcatggtcgatttctcccaggaaggcctcaggc

SEQ ID NO: 31 - DNA, 120 bp, Artificial Sequence
  Synthesized
actggtgcgtggactgggtgctagaattcatggtcgatttctcccaggaaggcctcaggcagaaggtgaagcaaaacaactgcttccaagagagactgggtgagacttgccaagtgacag

SEQ ID NO: 32 - DNA, 120 bp, Artificial Sequence
  Synthesized
tccaggttctttgggggcagaggggagttggggtgagggtgtctctctgtggctttacctgatgatcagggcttccatgaggaaatccagttcgtcctgttcagagcacacttcaggcag

SEQ ID NO: 33 - DNA, 120 bp, Artificial Sequence
  Synthesized
gatgatcagggcttccatgaggaaatccagttcgtcctgttcagagcacacttcaggcagcgtctgggcagagaaggggagggtggggaggaggaggaggctgtgagctgagaactgcag

SEQ ID NO: 34 - DNA, 120 bp, Artificial Sequence
  Synthesized
gagtccgcaagccaagggcaggctcaagagtgagccacttcttaccttcacagccacttgcagggggcttgggtcgttgggcattccggacacctggccttcatacacctccccaaaggc

SEQ ID NO: 35 - DNA, 120 bp, Artificial Sequence
  Synthesized
cagggggcttgggtcgttgggcattccggacacctggccttcatacacctccccaaaggcgccatggcccagacccctgtgcaaaggagaagacaagaggagacagagtcaaacaggcca

SEQ ID NO: 36 - DNA, 120 bp, Artificial Sequence
  Synthesized
ctcccaggacggcagcagggcgctcaccgaatgagggtgatgtttttccgcggcacctccttcaggtcactgatggaggaggtcttgccagcaaagcagtagttggggttgtagtcggtc

SEQ ID NO: 37 - DNA, 120 bp, Artificial Sequence
  Synthesized
ttcaggtcactgatggaggaggtcttgccagcaaagcagtagttggggttgtagtcggtcatgatggtcgaggtgcggagcttgctcagcttgtactcagggctctgcagctccatctgc

SEQ ID NO: 38 - DNA, 120 bp, Artificial Sequence
  Synthesized
atgatggtcgaggtgcggagcttgctcagcttgtactcagggctctgcagctccatctgcatggcttgcagctcctggtgcttccggcggtacactgcaggtgggtggtcagctgcaaca

SEQ ID NO: 39 - DNA, 120 bp, Artificial Sequence
  Synthesized
ctgcccccttgggagtccctggggctctgtgcactcaccaatcatgatgccggagaaagccaggaccagggcggccacgagggcagaggtcaccacagagaggatcagcgagagtggcag

SEQ ID NO: 40 - DNA, 120 bp, Artificial Sequence
  Synthesized
caggaccagggcggccacgagggcagaggtcaccacagagaggatcagcgagagtggcaggtgtggctccggggtgggtgacactggaagacaggtcccactggggtattgacaaccaca

SEQ ID NO: 41 - DNA, 120 bp, Artificial Sequence
  Synthesized
tcaggctatgggcccctctgcctcccctggccctgcccccttaccaatgcaggagacgccatcctcagccagcaccgtcccgtggtcacagaagcagatgaccttgtggctttcagggtc

SEQ ID NO: 42 - DNA, 120 bp, Artificial Sequence
  Synthesized
atcctcagccagcaccgtcccgtggtcacagaagcagatgaccttgtggctttcagggtccatgtgacattcgtctacctcacagtgactgcagtttagataatgcttaatattcacttc

SEQ ID NO: 43 - DNA, 120 bp, Artificial Sequence
  Synthesized
catgtgacattcgtctacctcacagtgactgcagtttagataatgcttaatattcacttccccgtggccttccatcactagtgacaaggagggagggtcagtcttgggccgagcctgcct

SEQ ID NO: 44 - DNA, 120 bp, Artificial Sequence
  Synthesized
gacctaagcaagtttgttctgctgcctggcagagaagctaccttttaaagctggggtgtacaggatgcccagtggactgatgaaggaaaccccatcttccccatccatttcggggtcatt

SEQ ID NO: 45 - DNA, 120 bp, Artificial Sequence
  Synthesized
caggatgcccagtggactgatgaaggaaaccccatcttccccatccatttcggggtcattgtttgaggctgcattgccgcctgagtagcaaaccagagcagagtttaacatggggggtgg

SEQ ID NO: 46 - DNA, 120 bp, Artificial Sequence
  Synthesized
gtgacaccttgaacacgaatcatctttacctatatatcctccgcctcctccacctgaggagcacccccctccaccccctccgaaaccccctcttgtctcccacccccacttcttcatggc

SEQ ID NO: 47 - DNA, 120 bp, Artificial Sequence
  Synthesized
gcacccccctccaccccctccgaaaccccctcttgtctcccacccccacttcttcatggcctgggggcaggaatgtcctccggtggcaccctcctgcaaagattttccggcccagagcaa

SEQ ID NO: 48 - DNA, 120 bp, Artificial Sequence
  Synthesized
ctgggggcaggaatgtcctccggtggcaccctcctgcaaagattttccggcccagagcaaggaagtgttatcattccagccacctccaccacctgcgggaagagatagggaacctgcgtg

SEQ ID NO: 49 - DNA, 120 bp, Artificial Sequence
  Synthesized
gaaggcctgggagaggttctgggagagggcacgcttgcagcgctttacctgcggctccggaattgccgtttagccctagaaccgaggagttattctccagtctctctgggtggaacgtgt

SEQ ID NO: 50 - DNA, 120 bp, Artificial Sequence
  Synthesized
aattgccgtttagccctagaaccgaggagttattctccagtctctctgggtggaacgtgtctgtcttggccccgtaggccctgccaccacctccggctgcaatgatcaggggcaccggca

SEQ ID NO: 51 - DNA, 120 bp, Artificial Sequence
  Synthesized
ctgtcttggccccgtaggccctgccaccacctccggctgcaatgatcaggggcaccggcactccatccttcatctgaccaggggagacattcagacattgagaaaccgagctgtgcttcc

SEQ ID NO: 52 - DNA, 120 bp, Artificial Sequence
  Synthesized
ctgacattgcagatgcacaggaacctggtggaaatctggcagcacacaccataccttaaatacgtaggtggctccaccccctcctcctccgcctcctgcccactcatgcacgcttctgtt

SEQ ID NO: 53 - DNA, 120 bp, Artificial Sequence
  Synthesized
tacgtaggtggctccaccccctcctcctccgcctcctgcccactcatgcacgcttctgttcacacggatttcttcttctatcacattgttctctccaatgcagactttctggattaactg

SEQ ID NO: 54 - DNA, 120 bp, Artificial Sequence
  Synthesized
cacacggatttcttcttctatcacattgttctctccaatgcagactttctggattaactggtttgtctgtagaaacaaaaagcacgttaggtttgtggccaaaccagagttctccacttt

SEQ ID NO: 55 - DNA, 120 bp, Artificial Sequence
  Synthesized
ggcctgacagagtgcagacgagaaacccctgctctgggcacttacactggggcaggcgtcctctccctgctgcccaaccaggatgtacagcatgtcatccttctccaggttgaagatgcc

SEQ ID NO: 56 - DNA, 120 bp, Artificial Sequence
  Synthesized
ctctccctgctgcccaaccaggatgtacagcatgtcatccttctccaggttgaagatgcccagcacagacacgccgtgggaccgcatcatggtgttcttcccgcctttcccgccagcagc

SEQ ID NO: 57 - DNA, 120 bp, Artificial Sequence
  Synthesized
cagcacagacacgccgtgggaccgcatcatggtgttcttcccgcctttcccgccagcagctccgtagcccgagatgctgcaatgggacaaagaacgttggctcccgctgtggtatgaaga

SEQ ID NO: 58 - DNA, 120 bp, Artificial Sequence
  Synthesized
gggtggtctgcccctcccctccccctcttccatacgcacctgtaggtgtcggtggctggcaccttccagatctggatgcctttcagggggccctcgctccccacctccacgctcaggttg

SEQ ID NO: 59 - DNA, 120 bp, Artificial Sequence
  Synthesized
accttccagatctggatgcctttcagggggccctcgctccccacctccacgctcaggttggagttctggtaggcgttgttgcactgtgcctgggtggggccatggggcccgctggcccca

SEQ ID NO: 60 - DNA, 120 bp, Artificial Sequence
  Synthesized
gagttctggtaggcgttgttgcactgtgcctgggtggggccatggggcccgctggccccacatgtggtgaacagccaatgaactgtggcacaagaggagaggcagtcactcatgtggcca

SEQ ID NO: 61 - DNA, 120 bp, Artificial Sequence
  Synthesized
ttctgccttttgcaacaagaagttactgtgctcacatttgtgagctgaacccttacctgtagggtcaaagatgggggtctgtcttggtgaattttccccgggtttcagctccttgtttgg

SEQ ID NO: 62 - DNA, 120 bp, Artificial Sequence
  Synthesized
agggtcaaagatgggggtctgtcttggtgaattttccccgggtttcagctccttgtttgggtttctctcaaacaggtttcttgatttgggtgctgtattctgcaggatcttgtcctctcc

SEQ ID NO: 63 - DNA, 120 bp, Artificial Sequence
  Synthesized
gtttctctcaaacaggtttcttgatttgggtgctgtattctgcaggatcttgtcctctccgctaactgcaatagagaagaccccacgggctgagttaggtgagggttgatttcagggtga

SEQ ID NO: 64 - DNA, 120 bp, Artificial Sequence
  Synthesized
atgggggttgggggacagagtgctggggtcagagtgaactcactggtgaggtagcagtccaggctgatggagatattgtcaaaagccacgatggctctggatccttgtccccaccatgcg

SEQ ID NO: 65 - DNA, 120 bp, Artificial Sequence
  Synthesized
aggctgatggagatattgtcaaaagccacgatggctctggatccttgtccccaccatgcgaccatctgcagccagaacctgtacacatcaagaggaatgtgtgtgaggagcaaactgggg

SEQ ID NO: 66 - DNA, 120 bp, Artificial Sequence
  Synthesized
ggagaagggtattgggggagatgcatagagcctacctgtcagacacatcgaggagaggcaacaccatccactgccacaggctcaagccttcataggcggcgacatgccagaccatcctgc

SEQ ID NO: 67 - DNA, 120 bp, Artificial Sequence
  Synthesized
acaccatccactgccacaggctcaagccttcataggcggcgacatgccagaccatcctgccttgctccttcccggttttgttctccactagcaccaaggacacgtttcccctcaagactc

SEQ ID NO: 68 - DNA, 120 bp, Artificial Sequence
  Synthesized
cttgctccttcccggttttgttctccactagcaccaaggacacgtttcccctcaagactccacgaatgagccaggacattcggagctgtgagggcgagaagagtcagaggacaaggtatg

SEQ ID NO: 69 - DNA, 120 bp, Artificial Sequence
  Synthesized
ggaggagaaattagagaactagagaaacaaggagacttgcctcacatggagagctcttgatcggtgcaggaaacgtagcactggtcactgtagcactttcagaagcggggacatcagtgg

SEQ ID NO: 70 - DNA, 120 bp, Artificial Sequence
  Synthesized
tcggtgcaggaaacgtagcactggtcactgtagcactttcagaagcggggacatcagtggtactgagcaatagagcatggtctaggagagaggaaaagaatcacaagcacgccattatca

SEQ ID NO: 71 - DNA, 120 bp, Artificial Sequence
  Synthesized
gtctatgtgggcatgaagatgggcaccagagagaaggcaggagagcagtagtaccttggtggtcctggaaccgggcatcctttagggtcctgacctgccattgaggagtgtggggtgaca

SEQ ID NO: 72 - DNA, 120 bp, Artificial Sequence
  Synthesized
ggtcctggaaccgggcatcctttagggtcctgacctgccattgaggagtgtggggtgacagtgtgccttgggtccagccacagaagccatcttcaaagttgcagtaaaaacccacaggca

SEQ ID NO: 73 - DNA, 120 bp, Artificial Sequence
  Synthesized
gtgtgccttgggtccagccacagaagccatcttcaaagttgcagtaaaaacccacaggcagtttccctatggagagagcagagaggcaccatcattttcaggaccactaaaggcaaaata

SEQ ID NO: 74 - DNA, 120 bp, Artificial Sequence
  Synthesized
gggttatgagcatgggctgggctcaggcagggtggggcagccccatctactcacggcacatctggctctcatcttctccctgggcacagtcctggtggaagtcacaggcctgcccaagct

SEQ ID NO: 75 - DNA, 120 bp, Artificial Sequence
  Synthesized
tctggctctcatcttctccctgggcacagtcctggtggaagtcacaggcctgcccaagctggaggactgtcccattccaacaagtgaaggagctctgcagggccatcttggagcctgggg

SEQ ID NO: 76 - DNA, 120 bp, Artificial Sequence
  Synthesized
ggaggactgtcccattccaacaagtgaaggagctctgcagggccatcttggagcctggggatgttcctggagagcacacagacacacaaccatggtaagtttgcatggccccaggcagca

SEQ ID NO: 77 - DNA, 120 bp, Artificial Sequence
  Synthesized
tgacacatctaacacaataggctaccaaggagcgtgggaaagccagattcagataccttcactgcagttcttcagggcaaagaagtccactgcagacaagctgcggtttccactggagat

SEQ ID NO: 78 - DNA, 120 bp, Artificial Sequence
  Synthesized
actgcagttcttcagggcaaagaagtccactgcagacaagctgcggtttccactggagatgtattccagggccactcgaaatgggttgtctggacgcccgattcttccctggagcactgt

SEQ ID NO: 79 - DNA, 120 bp, Artificial Sequence
  Synthesized
gtattccagggccactcgaaatgggttgtctggacgcccgattcttccctggagcactgtccaactggttgcattggaaaacagaggagaaaagcatagagaaacagatatgagaattag

SEQ ID NO: 80 - DNA, 120 bp, Artificial Sequence
  Synthesized
gacatggagaagtacttacccatgcttccctggagtgggcatcaggaggatctctcttgcagcctcgttgtggggcagcagctgggcaatgtaccttccagagggctgcaggtgcctgtg

SEQ ID NO: 81 - DNA, 120 bp, Artificial Sequence
  Synthesized
agcctcgttgtggggcagcagctgggcaatgtaccttccagagggctgcaggtgcctgtgcaccgagacggccagtgtgcagtgctcactgctgctcctcatccacggactcaggatggt

SEQ ID NO: 82 - DNA, 120 bp, Artificial Sequence
  Synthesized
caccgagacggccagtgtgcagtgctcactgctgctcctcatccacggactcaggatggtgtgcttggagtcagctgaggtgttgagaaggagaaaggagcctggaaagagacagggaaa

SEQ ID NO: 83 - DNA, 120 bp, Artificial Sequence
  Synthesized
tgacccacccaggacatcaccagcagcctctcccttacctctgggcatctccttagaacgctctgccccaggcccatccagcaagtccatctgggaggcctcctcggaggggatgcggcg

SEQ ID NO: 84 - DNA, 120 bp, Artificial Sequence
  Synthesized
ctctgccccaggcccatccagcaagtccatctgggaggcctcctcggaggggatgcggcgccaggaccagctctggttcctgaggtcatgcagtggaggggaatactccagctcacaggg

SEQ ID NO: 85 - DNA, 120 bp, Artificial Sequence
  Synthesized
ccaggaccagctctggttcctgaggtcatgcagtggaggggaatactccagctcacaggggaagtcaaagctgcactccagacctgcaataatagccaagggtcaatggaaaaaaccatt

SEQ ID NO: 86 - DNA, 120 bp, Artificial Sequence
  Synthesized
acagagtccttattatgagatagtgacagtgtatctcaagtaaatattaaacatatacttaccatatcggctgcgatgagacaggaaagggaaggagtctttcattatccaggtgagatt

SEQ ID NO: 87 - DNA, 120 bp, Artificial Sequence
  Synthesized
accatatcggctgcgatgagacaggaaagggaaggagtctttcattatccaggtgagattccatgtaaaataatcaggagaaggagaaggcatgtttgttggtgattccaaggagctatg

SEQ ID NO: 88 - DNA, 120 bp, Artificial Sequence
  Synthesized
ccatgtaaaataatcaggagaaggagaaggcatgtttgttggtgattccaaggagctatgacctggacataaaaataaagaaaacactgatccatgtgcttggggtgtgtcttttagctg

SEQ ID NO: 89 - DNA, 120 bp, Artificial Sequence
  Synthesized
cggcacactcaggcgggagctgctcaccagtcccgaagatctggaagagaaggcggggctgggaggcgcgaattgccgcggacagccttccctctctgcccacttccgacgccttcttct

SEQ ID NO: 90 - DNA, 120 bp, Artificial Sequence
  Synthesized
gggaggcgcgaattgccgcggacagccttccctctctgcccacttccgacgccttcttctcgggcatcaggcggatcctcagtcgcccttcgccttggcgaatccaccaactgaacagct

SEQ ID NO: 91 - DNA, 120 bp, Artificial Sequence
  Synthesized
cgggcatcaggcggatcctcagtcgcccttcgccttggcgaatccaccaactgaacagctcgctgagattgaactggagcagccccacagccgcctccccggggggcccgacgcaaccct

SEQ ID NO: 92 - DNA, 120 bp, Artificial Sequence
  Synthesized
cgctgagattgaactggagcagccccacagccgcctccccggggggcccgacgcaaccctccaagatcgcctcctcgcccagctccagcaccaactgcttggcacgccggagcttgcgca

SEQ ID NO: 93 - DNA, 120 bp, Artificial Sequence
  Synthesized
ccaagatcgcctcctcgcccagctccagcaccaactgcttggcacgccggagcttgcgcacggagccgcccttcagcaccctggacagcgtccgggcctctgccggggctggtgaaccgg

SEQ ID NO: 94 - DNA, 120 bp, Artificial Sequence
  Synthesized
cggagccgcccttcagcaccctggacagcgtccgggcctctgccggggctggtgaaccggcggtccaggagacccccggcgccggccccagcaacctgagcagcggggcgcagtccagag

SEQ ID NO: 95 - DNA, 120 bp, Artificial Sequence
  Synthesized
cggtccaggagacccccggcgccggccccagcaacctgagcagcggggcgcagtccagagctagcgagccgcgggcctcgggcctgccagccttcagctccgaggaggatggtggcagca

SEQ ID NO: 96 - DNA, 120 bp, Artificial Sequence
  Synthesized
ctagcgagccgcgggcctcgggcctgccagccttcagctccgaggaggatggtggcagcagtaggtcccgggcgtagacacggaagagcgagggcaccacgaagtcaactgccagactct

SEQ ID NO: 97 - DNA, 120 bp, Artificial Sequence
  Synthesized
gtaggtcccgggcgtagacacggaagagcgagggcaccacgaagtcaactgccagactcttcctctgcaggcgcgagtagctgagtggctcccggggctgcagcggcggccccgcagctg

SEQ ID NO: 98 - DNA, 120 bp, Artificial Sequence
  Synthesized
tcctctgcaggcgcgagtagctgagtggctcccggggctgcagcggcggccccgcagctggggagcccgcgcgctggccggtccccatcccggagcccacagctgccgtggaaagcagca

SEQ ID NO: 99 - DNA, 120 bp, Artificial Sequence
  Synthesized
gggagcccgcgcgctggccggtccccatcccggagcccacagctgccgtggaaagcagcagcggcaggagccacaggagcccgatggctcccatcccgccggaggaggccgtttacactg

SEQ ID NO: 100 - DNA, 120 bp, Artificial Sequence
  Synthesized
cccctgactccgtccagtattgatcgggagagccggagcgagctcttcggggagcagcgatgcgaccctccgggacggccggggcagcgctcctggcgctgctggctgcgctctgcccgg

SEQ ID NO: 101 - DNA, 120 bp, Artificial Sequence
  Synthesized
tgcgaccctccgggacggccggggcagcgctcctggcgctgctggctgcgctctgcccggcgagtcgggctctggaggaaaagaaaggtaagggcgtgtctcgccggctcccgcgccgcc

SEQ ID NO: 102 - DNA, 120 bp, Artificial Sequence
  Synthesized
tgcatttctcagtatttcatgtgatatctgtctttttcttccagtttgccaaggcacgagtaacaagctcacgcagttgggcacttttgaagatcattttctcagcctccagaggatgtt

SEQ ID NO: 103 - DNA, 120 bp, Artificial Sequence
  Synthesized
taacaagctcacgcagttgggcacttttgaagatcattttctcagcctccagaggatgttcaataactgtgaggtggtccttgggaatttggaaattacctatgtgcagaggaattatga

SEQ ID NO: 104 - DNA, 120 bp, Artificial Sequence
  Synthesized
caataactgtgaggtggtccttgggaatttggaaattacctatgtgcagaggaattatgatctttccttcttaaaggttggtgactttgattttcctacacaaataaaattggagaaaat

SEQ ID NO: 105 - DNA, 120 bp, Artificial Sequence
  Synthesized
atcacgcatttatgttttctcttcttagaccatccaggaggtggctggttatgtcctcattgccctcaacacagtggagcgaattcctttggaaaacctgcagatcatcagaggaaatat

SEQ ID NO: 106 - DNA, 120 bp, Artificial Sequence
  Synthesized
tgccctcaacacagtggagcgaattcctttggaaaacctgcagatcatcagaggaaatatgtactacgaaaattcctatgccttagcagtcttatctaactatgatgcaaataaaaccgg

SEQ ID NO: 107 - DNA, 120 bp, Artificial Sequence
  Synthesized
gtactacgaaaattcctatgccttagcagtcttatctaactatgatgcaaataaaaccggactgaaggagctgcccatgagaaatttacagggtgagaggctgggatgccaaggctgggg

SEQ ID NO: 108 - DNA, 120 bp, Artificial Sequence
  Synthesized
gggaatttaaaggagctggaaagagtgctcaccgcagttccattctcccgcagaaatcctgcatggcgccgtgcggttcagcaacaaccctgccctgtgcaacgtggagagcatccagtg

SEQ ID NO: 109 - DNA, 120 bp, Artificial Sequence
  Synthesized
gcatggcgccgtgcggttcagcaacaaccctgccctgtgcaacgtggagagcatccagtggcgggacatagtcagcagtgactttctcagcaacatgtcgatggacttccagaaccacct

SEQ ID NO: 110 - DNA, 120 bp, Artificial Sequence
  Synthesized
gcgggacatagtcagcagtgactttctcagcaacatgtcgatggacttccagaaccacctgggcagctgtaagtgtcgcatacacactatctctgcctccagctcctatgggggacagct

SEQ ID NO: 111 - DNA, 120 bp, Artificial Sequence
  Synthesized
gtcatcagtttctcatcatttcactgagatatgcatctattacttttacatttcaggccaaaagtgtgatccaagctgtcccaatgggagctgctggggtgcaggagaggagaactgcca

SEQ ID NO: 112 - DNA, 120 bp, Artificial Sequence
  Synthesized
aaagtgtgatccaagctgtcccaatgggagctgctggggtgcaggagaggagaactgccagaaacgtaagtcagtgaacagcctcagacccatgtgtgaccgcccctctcttccttcact

SEQ ID NO: 113 - DNA, 120 bp, Artificial Sequence
  Synthesized
cagctcacagggaacctttgctctttttcagtgaccaaaatcatctgtgcccagcagtgctccgggcgctgccgtggcaagtcccccagtgactgctgccacaaccagtgtgctgcaggc

SEQ ID NO: 114 - DNA, 120 bp, Artificial Sequence
  Synthesized
tccgggcgctgccgtggcaagtcccccagtgactgctgccacaaccagtgtgctgcaggctgcacaggcccccgggagagcgactgcctggtaagatgcccctccagcagcctccctgga

SEQ ID NO: 115 - DNA, 120 bp, Artificial Sequence
  Synthesized
gctgagtgtacttacctcacttgcccagcgtgtcctctctcctccataggtctgccgcaaattccgagacgaagccacgtgcaaggacacctgccccccactcatgctctacaaccccac

SEQ ID NO: 116 - DNA, 120 bp, Artificial Sequence
  Synthesized
attccgagacgaagccacgtgcaaggacacctgccccccactcatgctctacaaccccaccacgtaccagatggatgtgaaccccgagggcaaatacagctttggtgccacctgcgtgaa

SEQ ID NO: 117 - DNA, 120 bp, Artificial Sequence
  Synthesized
cacgtaccagatggatgtgaaccccgagggcaaatacagctttggtgccacctgcgtgaagaagtgtccccgtgagtcctcctctgtgggccctctaactggtcaggcatccttgtcccg

SEQ ID NO: 118 - DNA, 120 bp, Artificial Sequence
  Synthesized
tcatcaccttcctttcatgctctcttccccaggtaattatgtggtgacagatcacggctcgtgcgtccgagcctgtggggccgacagctatgagatggaggaagacggcgtccgcaagtg

SEQ ID NO: 119 - DNA, 120 bp, Artificial Sequence
  Synthesized
gtgcgtccgagcctgtggggccgacagctatgagatggaggaagacggcgtccgcaagtgtaagaagtgcgaagggccttgccgcaaaggtaggaagcccgccggtgtgcggacgaggct

SEQ ID NO: 120 - DNA, 120 bp, Artificial Sequence
  Synthesized
tagctattcttaatccaacaaatgtgaacggaatacacgtctctcttatctctgcagtgtgtaacggaataggtattggtgaatttaaagactcactctccataaatgctacgaatatta

SEQ ID NO: 121 - DNA, 120 bp, Artificial Sequence
  Synthesized
gtaacggaataggtattggtgaatttaaagactcactctccataaatgctacgaatattaaacacttcaaaaactgcacctccatcagtggcgatctccacatcctgccggtggcattta

SEQ ID NO: 122 - DNA, 120 bp, Artificial Sequence
  Synthesized
aacacttcaaaaactgcacctccatcagtggcgatctccacatcctgccggtggcatttagggggtgagtcacaggttcagttgcttgtataaagaaaaacaaaatctgcctttttaact

SEQ ID NO: 123 - DNA, 120 bp, Artificial Sequence
  Synthesized
taactggtagagattggtgatcaataatcaccctgttgtttgtttcagtgactccttcacacatactcctcctctggatccacaggaactggatattctgaaaaccgtaaaggaaatcac

SEQ ID NO: 124 - DNA, 120 bp, Artificial Sequence
  Synthesized
acatactcctcctctggatccacaggaactggatattctgaaaaccgtaaaggaaatcacaggtttgagctgaattatcacatgaatataaatgggaaatcagtgttttagagagagaac

SEQ ID NO: 125 - DNA, 120 bp, Artificial Sequence
  Synthesized
aactcctacgtggtgtgtgtctgaagtctttcatctgccttacagggtttttgctgattcaggcttggcctgaaaacaggacggacctccatgcctttgagaacctagaaatcatacgcg

SEQ ID NO: 126 - DNA, 120 bp, Artificial Sequence
  Synthesized
aggcttggcctgaaaacaggacggacctccatgcctttgagaacctagaaatcatacgcggcaggaccaagcaacagtaagttgaccacagccaaagcctggtagattacatttgccttt

SEQ ID NO: 127 - DNA, 120 bp, Artificial Sequence
  Synthesized
ttttcttctctccaatgtagtggtcagttttctcttgcagtcgtcagcctgaacataacatccttgggattacgctccctcaaggagataagtgatggagatgtgataatttcaggaaac

SEQ ID NO: 128 - DNA, 120 bp, Artificial Sequence
  Synthesized
tccttgggattacgctccctcaaggagataagtgatggagatgtgataatttcaggaaacaaaaatttgtgctatgcaaatacaataaactggaaaaaactgtttgggacctccggtcag

SEQ ID NO: 129 - DNA, 120 bp, Artificial Sequence
  Synthesized
aaaaatttgtgctatgcaaatacaataaactggaaaaaactgtttgggacctccggtcagaaaaccaaaattataagcaacagaggtgaaaacagctgcagtaagtcaccgctttctgtt

SEQ ID NO: 130 - DNA, 120 bp, Artificial Sequence
  Synthesized
cccctcgggtccctgctctgtcactgactgctgtgacccactctgtctccgcagaggccacaggccaggtctgccatgccttgtgctcccccgagggctgctggggcccggagcccaggg

SEQ ID NO: 131 - DNA, 120 bp, Artificial Sequence
  Synthesized
caggccaggtctgccatgccttgtgctcccccgagggctgctggggcccggagcccagggactgcgtctcttgccggaatgtcagccgaggcagggaatgcgtggacaagtgcaaccttc

SEQ ID NO: 132 - DNA, 120 bp, Artificial Sequence
  Synthesized
actgcgtctcttgccggaatgtcagccgaggcagggaatgcgtggacaagtgcaaccttctggaggggtaggaggttatttctttaatccccttgcgttgatcaaaaataaggctccagg

SEQ ID NO: 133 - DNA, 120 bp, Artificial Sequence
  Synthesized
aatgtctcaggggtgggctgacgggtttcctcttcctcctctcagtgagccaagggagtttgtggagaactctgagtgcatacagtgccacccagagtgcctgcctcaggccatgaacat

SEQ ID NO: 134 - DNA, 120 bp, Artificial Sequence
  Synthesized
tgtggagaactctgagtgcatacagtgccacccagagtgcctgcctcaggccatgaacatcacctgcacaggacgggtaagagccccttgctgctatccacgtccatttcatgggaaggg

SEQ ID NO: 135 - DNA, 120 bp, Artificial Sequence
  Synthesized
gaaagagacatgcatgaacatttttctccaccttggtgcagggaccagacaactgtatccagtgtgcccactacattgacggcccccactgcgtcaagacctgcccggcaggagtcatgg

SEQ ID NO: 136 - DNA, 120 bp, Artificial Sequence
  Synthesized
agtgtgcccactacattgacggcccccactgcgtcaagacctgcccggcaggagtcatgggagaaaacaacaccctggtctggaagtacgcagacgccggccatgtgtgccacctgtgcc

SEQ ID NO: 137 - DNA, 120 bp, Artificial Sequence
  Synthesized
gagaaaacaacaccctggtctggaagtacgcagacgccggccatgtgtgccacctgtgccatccaaactgcacctacgggtgagtggaaagtgaaggagaacagaacatttcctctcttg

SEQ ID NO: 138 - DNA, 120 bp, Artificial Sequence
  Synthesized
gccaaatatagaaagaggggatttagtcaagatttaaattaaaaatgttagtggtcatttttctaatgtctttctattttttcccaggtcctaataaatcttcactgtctgactttagtc

SEQ ID NO: 139 - DNA, 120 bp, Artificial Sequence
  Synthesized
ttctaatgtctttctattttttcccaggtcctaataaatcttcactgtctgactttagtctcccactaaaactgcatttcctttctacaatttcaatttctccctttgcttcaaataaag

SEQ ID NO: 140 - DNA, 120 bp, Artificial Sequence
  Synthesized
ggccaggaaatgagagtctcaaagccatgttattctgcctttttaaactatcatcctgtaatcaaagtaatgatggcagcgtgtcccaccagagcgggagcccagctgctcaggagtcat

SEQ ID NO: 141 - DNA, 120 bp, Artificial Sequence
  Synthesized
atcaaagtaatgatggcagcgtgtcccaccagagcgggagcccagctgctcaggagtcatgcttaggatggatcccttctcttctgccgtcagagtttcagctgggttggggtggatgca

SEQ ID NO: 142 - DNA, 120 bp, Artificial Sequence
  Synthesized
gcttaggatggatcccttctcttctgccgtcagagtttcagctgggttggggtggatgcagccacctccatgcctggccttctgcatctgtgatcatcacggcctcctcctgccactgag

SEQ ID NO: 143 - DNA, 120 bp, Artificial Sequence
  Synthesized
taaaaatctccaaaatatatgccaaagaagtagaatgagaaaaatgtatatttctctttcacttcctacagatgcactgggccaggtcttgaaggctgtccaacgaatgggtaagtgttc

SEQ ID NO: 144 - DNA, 120 bp, Artificial Sequence
  Synthesized
acttcctacagatgcactgggccaggtcttgaaggctgtccaacgaatgggtaagtgttcacagctctgtgtcacatggacctcgtcaagaatgaccacactgctgtgggtgaagatgct

SEQ ID NO: 145 - DNA, 120 bp, Artificial Sequence
  Synthesized
gactggggagagcttgagaaagttggaaacgttgccttagaagcctgttttttctccttttagaagctacatagtgtctcactttccaagatcattctacaagatgtcagtgcactgaaa

SEQ ID NO: 146 - DNA, 120 bp, Artificial Sequence
  Synthesized
tagaagctacatagtgtctcactttccaagatcattctacaagatgtcagtgcactgaaacatgcaggggcgtgttgagtgccaaggccatggaatctgtcagcaacctcacccttcctt

SEQ ID NO: 147 - DNA, 120 bp, Artificial Sequence
  Synthesized
ggaatctgtcagcaacctcacccttccttgttcctccacctcattccaggcctaagatcccgtccatcgccactgggatggtgggggccctcctcttgctgctggtggtggccctgggga

SEQ ID NO: 148 - DNA, 120 bp, Artificial Sequence
  Synthesized
cgtccatcgccactgggatggtgggggccctcctcttgctgctggtggtggccctggggatcggcctcttcatgcgaaggcgccacatcgttcggaagcgcacgctgcggaggctgctgc

SEQ ID NO: 149 - DNA, 120 bp, Artificial Sequence
  Synthesized
tcggcctcttcatgcgaaggcgccacatcgttcggaagcgcacgctgcggaggctgctgcaggagagggaggtgagtgccagtcctgggtgggctcaggagccctcgcaccccgacagga

SEQ ID NO: 150 - DNA, 120 bp, Artificial Sequence
  Synthesized
gctttccagcatggtgagggctgaggtgacccttgtctctgtgttcttgtcccccccagcttgtggagcctcttacacccagtggagaagctcccaaccaagctctcttgaggatcttga

SEQ ID NO: 151 - DNA, 120 bp, Artificial Sequence
  Synthesized
ttgtggagcctcttacacccagtggagaagctcccaaccaagctctcttgaggatcttgaaggaaactgaattcaaaaagatcaaagtgctgggctccggtgcgttcggcacggtgtata

SEQ ID NO: 152 - DNA, 120 bp, Artificial Sequence
  Synthesized
aggaaactgaattcaaaaagatcaaagtgctgggctccggtgcgttcggcacggtgtataaggtaaggtccctggcacaggcctctgggctgggccgcagggcctctcatggtctggtgg

SEQ ID NO: 153 - DNA, 120 bp, Artificial Sequence
  Synthesized
cacaattgccagttaacgtcttccttctctctctgtcatagggactctggatcccagaaggtgagaaagttaaaattcccgtcgctatcaaggaattaagagaagcaacatctccgaaag

SEQ ID NO: 154 - DNA, 120 bp, Artificial Sequence
  Synthesized
gtgagaaagttaaaattcccgtcgctatcaaggaattaagagaagcaacatctccgaaagccaacaaggaaatcctcgatgtgagtttctgctttgctgtgtgggggtccatggctctga

SEQ ID NO: 155 - DNA, 120 bp, Artificial Sequence
  Synthesized
cactgacgtgcctctccctccctccaggaagcctacgtgatggccagcgtggacaacccccacgtgtgccgcctgctgggcatctgcctcacctccaccgtgcagctcatcacgcagctc

SEQ ID NO: 156 - DNA, 120 bp, Artificial Sequence
  Synthesized
cacgtgtgccgcctgctgggcatctgcctcacctccaccgtgcagctcatcacgcagctcatgcccttcggctgcctcctggactatgtccgggaacacaaagacaatattggctcccag

SEQ ID NO: 157 - DNA, 120 bp, Artificial Sequence
  Synthesized
atgcccttcggctgcctcctggactatgtccgggaacacaaagacaatattggctcccagtacctgctcaactggtgtgtgcagatcgcaaaggtaatcagggaagggagatacggggag

SEQ ID NO: 158 - DNA, 120 bp, Artificial Sequence
  Synthesized
catgatgatctgtccctcacagcagggtcttctctgtttcagggcatgaactacttggaggaccgtcgcttggtgcaccgcgacctggcagccaggaacgtactggtgaaaacaccgcag

SEQ ID NO: 159 - DNA, 120 bp, Artificial Sequence
  Synthesized
gaccgtcgcttggtgcaccgcgacctggcagccaggaacgtactggtgaaaacaccgcagcatgtcaagatcacagattttgggctggccaaactgctgggtgcggaagagaaagaatac

SEQ ID NO: 160 - DNA, 120 bp, Artificial Sequence
  Synthesized
catgtcaagatcacagattttgggctggccaaactgctgggtgcggaagagaaagaataccatgcagaaggaggcaaagtaaggaggtggctttaggtcagccagcattttcctgacacc

SEQ ID NO: 161 - DNA, 120 bp, Artificial Sequence
  Synthesized
aacagagggaaactaatagttgtctcactgcctcatctctcaccatcccaaggtgcctatcaagtggatggcattggaatcaattttacacagaatctatacccaccagagtgatgtctg

SEQ ID NO: 162 - DNA, 120 bp, Artificial Sequence
  Synthesized
caagtggatggcattggaatcaattttacacagaatctatacccaccagagtgatgtctggagctacggtgagtcataatcctgatgctaatgagtttgtactgaggccaagctggcttt

SEQ ID NO: 163 - DNA, 120 bp, Artificial Sequence
  Synthesized
gttcattcatgatcccactgccttcttttcttgcttcatcctctcaggggtgactgtttgggagttgatgacctttggatccaagccatatgacggaatccctgccagcgagatctcctc

SEQ ID NO: 164 - DNA, 120 bp, Artificial Sequence
  Synthesized
ggagttgatgacctttggatccaagccatatgacggaatccctgccagcgagatctcctccatcctggagaaaggagaacgcctccctcagccacccatatgtaccatcgatgtctacat

SEQ ID NO: 165 - DNA, 120 bp, Artificial Sequence
  Synthesized
catcctggagaaaggagaacgcctccctcagccacccatatgtaccatcgatgtctacatgatcatggtcaagtgtgagtgactggtgggtctgtccacactgcctagctgagccttggt

SEQ ID NO: 166 - DNA, 120 bp, Artificial Sequence
  Synthesized
cagtgttctaattgcactgttttttctcattccttccccaggctggatgatagacgcagatagtcgcccaaagttccgtgagttgatcatcgaattctccaaaatggcccgagaccccca

SEQ ID NO: 167 - DNA, 120 bp, Artificial Sequence
  Synthesized
tagtcgcccaaagttccgtgagttgatcatcgaattctccaaaatggcccgagacccccagcgctaccttgtcattcaggtacaaattgcagtctgtgcttccattgggaagagtccctc

SEQ ID NO: 168 - DNA, 120 bp, Artificial Sequence
  Synthesized
cgggccattctaatagcctcaaaatctctgcaccagggggatgaaagaatgcatttgccaagtcctacagactccaacttctaccgtgccctgatggatgaagaagacatggacgacgtg

SEQ ID NO: 169 - DNA, 120 bp, Artificial Sequence
  Synthesized
agtcctacagactccaacttctaccgtgccctgatggatgaagaagacatggacgacgtggtggatgccgacgagtacctcatcccacagcagggcttcttcagcagcccctccacgtca

SEQ ID NO: 170 - DNA, 120 bp, Artificial Sequence
  Synthesized
gtggatgccgacgagtacctcatcccacagcagggcttcttcagcagcccctccacgtcacggactcccctcctgagctctctggtatgaaatctctgtctctctctctctctcaagctg

SEQ ID NO: 171 - DNA, 120 bp, Artificial Sequence
  Synthesized
attcaggaaaagtggatgagatgtggtacaagcattccatgggcaacttctctgtttctttttcagagtgcaaccagcaacaattccaccgtggcttgcattgatagaaatggggtatgt

SEQ ID NO: 172 - DNA, 120 bp, Artificial Sequence
  Synthesized
tttcagagtgcaaccagcaacaattccaccgtggcttgcattgatagaaatggggtatgtatgaacaccttataagccagaatttacagctctccactatggctctattttacatggaaa

SEQ ID NO: 173 - DNA, 120 bp, Artificial Sequence
  Synthesized
taaccttccctcatttcctcctgcagctgcaaagctgtcccatcaaggaagacagcttcttgcagcgatacagctcagaccccacaggcgccttgactgaggacagcatagacgacacct

SEQ ID NO: 174 - DNA, 120 bp, Artificial Sequence
  Synthesized
tgcagcgatacagctcagaccccacaggcgccttgactgaggacagcatagacgacaccttcctcccagtgcctggtgagtggcttgtctggaaacagtcctgctcctcaacctcctcga

SEQ ID NO: 175 - DNA, 120 bp, Artificial Sequence
  Synthesized
tcctcccagtgcctggtgagtggcttgtctggaaacagtcctgctcctcaacctcctcgacccactcagcagcagccagtctccagtgtccaagccaggtgctccctccagcatctccag

SEQ ID NO: 176 - DNA, 120 bp, Artificial Sequence
  Synthesized
cccactcagcagcagccagtctccagtgtccaagccaggtgctccctccagcatctccagagggggaaacagtggcagatttgcagacacagtgaagggcgtaaggagcagataaacaca

SEQ ID NO: 177 - DNA, 120 bp, Artificial Sequence
  Synthesized
tcacctctgatttctttccactttcagaatacataaaccagtccgttcccaaaaggcccgctggctctgtgcagaatcctgtctatcacaatcagcctctgaaccccgcgcccagcagag

SEQ ID NO: 178 - DNA, 120 bp, Artificial Sequence
  Synthesized
ctggctctgtgcagaatcctgtctatcacaatcagcctctgaaccccgcgcccagcagagacccacactaccaggacccccacagcactgcagtgggcaaccccgagtatctcaacactg

SEQ ID NO: 179 - DNA, 120 bp, Artificial Sequence
  Synthesized
acccacactaccaggacccccacagcactgcagtgggcaaccccgagtatctcaacactgtccagcccacctgtgtcaacagcacattcgacagccctgcccactgggcccagaaaggca

SEQ ID NO: 180 - DNA, 120 bp, Artificial Sequence
  Synthesized
tccagcccacctgtgtcaacagcacattcgacagccctgcccactgggcccagaaaggcagccaccaaattagcctggacaaccctgactaccagcaggacttctttcccaaggaagcca

SEQ ID NO: 181 - DNA, 120 bp, Artificial Sequence
  Synthesized
gccaccaaattagcctggacaaccctgactaccagcaggacttctttcccaaggaagccaagccaaatggcatctttaagggctccacagctgaaaatgcagaatacctaagggtcgcgc

SEQ ID NO: 182 - DNA, 120 bp, Artificial Sequence
  Synthesized
agccaaatggcatctttaagggctccacagctgaaaatgcagaatacctaagggtcgcgccacaaagcagtgaatttattggagcatgaccacggaggatagtatgagccctaaaaatcc

SEQ ID NO: 183 - DNA, 120 bp, Artificial Sequence
  Synthesized
acagaactgtgttttgatgttaacaaattgtacgaacacaagacttaagaaataagagcagatgctgccatgatggtggctacttgaaggctgcaaattctcctgtagagggaggacaag

SEQ ID NO: 184 - DNA, 120 bp, Artificial Sequence
  Synthesized
gatgctgccatgatggtggctacttgaaggctgcaaattctcctgtagagggaggacaagagctaattttaaaaaagtcatctcaaataacctagagacacagaaaagcctcatttgagc

SEQ ID NO: 185 - DNA, 120 bp, Artificial Sequence
  Synthesized
ttccttttgttgctactctcctgaactctctcactcatttgtttcagtggacaggaaacgcaccatatccccctgcctggatgggtgtttttggagaagcacaagcatatagactaaaat

SEQ ID NO: 186 - DNA, 120 bp, Artificial Sequence
  Synthesized
caccatatccccctgcctggatgggtgtttttggagaagcacaagcatatagactaaaatcctctgtttggaaaccagcccgattcaaggagggttctgatgcactgcggtgaatttttg

SEQ ID NO: 187 - DNA, 120 bp, Artificial Sequence
  Synthesized
cctctgtttggaaaccagcccgattcaaggagggttctgatgcactgcggtgaatttttggcaatgagcgggccagcagctcaatagaggcgagaatctacaaaaaaaaaaagaaaaaaa

SEQ ID NO: 188 - DNA, 120 bp, Artificial Sequence
  Synthesized
tctgtctatgaatgttagtctgttcttttggatagcatgaagcttttacttacttggggaaagagtggtctctcatctcttttctttttgaggcactctgccattaatctcttcatggct

SEQ ID NO: 189 - DNA, 120 bp, Artificial Sequence
  Synthesized
aagagtggtctctcatctcttttctttttgaggcactctgccattaatctcttcatggcttttggacagttactccgtaccttactgagatctggagacaggtatcctcgtcccaccata

SEQ ID NO: 190 - DNA, 120 bp, Artificial Sequence
  Synthesized
tttggacagttactccgtaccttactgagatctggagacaggtatcctcgtcccaccataaaaattatctggagagagaaaaaaaagggaaataattcaaccttgtagataagttgaaaa

SEQ ID NO: 191 - DNA, 120 bp, Artificial Sequence
  Synthesized
aagaattaactagtaaaggccttacccttctgttggtcacacattcctctcatggagagtaagcccttgccacatcatttattatactcccatggaggacaaagcacctaaaatgccaag

SEQ ID NO: 192 - DNA, 120 bp, Artificial Sequence
  Synthesized
aagcccttgccacatcatttattatactcccatggaggacaaagcacctaaaatgccaagaagtggatgagagaagaactctgggatgcttttatctgtattttggagataatacttgtt

SEQ ID NO: 193 - DNA, 120 bp, Artificial Sequence
  Synthesized
tggagccttgtatatagacggtaaaataaacaccaagacgtggtaaatatttacctggtccctgttgttgatgtttgaataaggtaactgtccagtcatcaattcatacagaacaattcc

SEQ ID NO: 194 - DNA, 120 bp, Artificial Sequence
  Synthesized
cctgttgttgatgtttgaataaggtaactgtccagtcatcaattcatacagaacaattccaaatgcatatacatctgactgaaagctgtatggatttttatcttgcattctgatgacttc

SEQ ID NO: 195 - DNA, 120 bp, Artificial Sequence
  Synthesized
aaatgcatatacatctgactgaaagctgtatggatttttatcttgcattctgatgacttctggtgcctgttagaacatacaaagaaaaatattcttcacttcaattgaataaagactgaa

SEQ ID NO: 196 - DNA, 120 bp, Artificial Sequence
  Synthesized
taatcagtggaaaaatagcctcaattcttaccatccacaaaatggatccagacaactgttcaaactgatgggacccactccatcgagatttcactgtagctagaccaaaatcacctattt

SEQ ID NO: 197 - DNA, 120 bp, Artificial Sequence
  Synthesized
caaactgatgggacccactccatcgagatttcactgtagctagaccaaaatcacctatttttactgtgaggtcttcatgaagaaatatatctgaggtgtagtaagtaaaggaaaacagta

SEQ ID NO: 198 - DNA, 120 bp, Artificial Sequence
  Synthesized
aagacaaaatgcagaagaaaaagtcaggatgttttcaaacttcgcagacaaatttcaggaaggatactattactcttgaggtctctgtggatgattgacttggcgtgtaagtaactgaaa

SEQ ID NO: 199 - DNA, 120 bp, Artificial Sequence
  Synthesized
aggatactattactcttgaggtctctgtggatgattgacttggcgtgtaagtaactgaaaaacaaaacatcattttaacctgagtagggctaaaggactctggcctcgaaatctacagaa

SEQ ID NO: 200 - DNA, 120 bp, Artificial Sequence
  Synthesized
aatttacaagacatttaacgaatggaacttactccatgccctgtgcagtctgtcgtgcaatatctataagtttgatcatctcaaatttggtctcaatgatatggagatggtgatacaagc

SEQ ID NO: 201 - DNA, 120 bp, Artificial Sequence
  Synthesized
tatctataagtttgatcatctcaaatttggtctcaatgatatggagatggtgatacaagctggagccctcacaccactgggtaacaatagccagttgtggctttgtggaatagcccatga

SEQ ID NO: 202 - DNA, 120 bp, Artificial Sequence
  Synthesized
tggagccctcacaccactgggtaacaatagccagttgtggctttgtggaatagcccatgaagagtaggatattcacatgtcgtgttttcctgtacaaagaaatgtgacagtaaacattaa

SEQ ID NO: 203 - DNA, 120 bp, Artificial Sequence
  Synthesized
ataaaaataacttctttctctggaaaagagtaattcacacaagctcacctgagtactcctacttcatttttgaaggcttgtaactgctgaggtgtaggtgctgtcacattcaacattttc

SEQ ID NO: 204 - DNA, 120 bp, Artificial Sequence
  Synthesized
acttcatttttgaaggcttgtaactgctgaggtgtaggtgctgtcacattcaacattttcactgccacatcacctaaaaggcaattgttactccaagtgtcatttcaatttttaaaattt

SEQ ID NO: 205 - DNA, 120 bp, Artificial Sequence
  Synthesized
ttgtcacaatgtcaccacattacatacttaccatgccactttcccttgtagactgttccaaatgatccagatccaattctttgtcccactgtaatctgcccatcaggaatctcccaatca

SEQ ID NO: 206 - DNA, 120 bp, Artificial Sequence
  Synthesized
aatgatccagatccaattctttgtcccactgtaatctgcccatcaggaatctcccaatcatcactcgagtcccgtctaccaagtgttttcttgataaaaacagtaaaaaagtcaagtcaa

SEQ ID NO: 207 - DNA, 120 bp, Artificial Sequence
  Synthesized
tgacttctaagaagaaagaattcagagaaaaaaagatatcatatactcttaccattcgattcctgtcttctgaggatgaagatgacttcctttctcgctgaggtcctggagatttctgta

SEQ ID NO: 208 - DNA, 120 bp, Artificial Sequence
  Synthesized
tcctgtcttctgaggatgaagatgacttcctttctcgctgaggtcctggagatttctgtaaggctttcacgttagttagtgagccaggtaatgaggcagggggggtagcagacaaacctg

SEQ ID NO: 209 - DNA, 120 bp, Artificial Sequence
  Synthesized
aggctttcacgttagttagtgagccaggtaatgaggcagggggggtagcagacaaacctgtggttgatcctaaattagtgaaaagaaaaatgtatacattaaggaggagcaagtatgtta

SEQ ID NO: 210 - DNA, 120 bp, Artificial Sequence
  Synthesized
tataaaggaaataagcagcaaagcaattgcagtttccttgagtttttaaaaaaacctgaaatcactacttacctccatcaccacgaaatccttggtctctaatcaagtcctacaaataaa

SEQ ID NO: 211 - DNA, 120 bp, Artificial Sequence
  Synthesized
atcactacttacctccatcaccacgaaatccttggtctctaatcaagtcctacaaataaatagtaatgtatatttattccaagcaagcatataatcagagagtagcgataacactgaatt

SEQ ID NO: 212 - DNA, 120 bp, Artificial Sequence
  Synthesized
acttggtttttttttagttctagcaatgctggatacttacatcaatattgacaggttctattgtgtttatatgcacattgggagctgatgaggatcggtctcgttgcccaaattgatttc

SEQ ID NO: 213 - DNA, 120 bp, Artificial Sequence
  Synthesized
ttgtgtttatatgcacattgggagctgatgaggatcggtctcgttgcccaaattgatttcgatgatcttcatctgctggtcggaagggctgtggaattggaatggattttgaaggagacg

SEQ ID NO: 214 - DNA, 120 bp, Artificial Sequence
  Synthesized
gatgatcttcatctgctggtcggaagggctgtggaattggaatggattttgaaggagacggactggtgagaatttggggcctggaaaaatgaagtcattggaagataagattcagagtaa

SEQ ID NO: 215 - DNA, 120 bp, Artificial Sequence
  Synthesized
tgtcgcccaagagcagaagtcaaaccatacccaatagagtccgaggcgggtgcggaaggggatgatccagatgttagggcagtctctgctaaggacgcctcttcctgtggtattgggtgg

SEQ ID NO: 216 - DNA, 120 bp, Artificial Sequence
  Synthesized
gatgatccagatgttagggcagtctctgctaaggacgcctcttcctgtggtattgggtggtgttcaaagaacttggagacaaacagcaaactgtgaggcaaaacaaaacaaacctaactt

SEQ ID NO: 217 - DNA, 120 bp, Artificial Sequence
  Synthesized
aagtgtaaaatggtaggtagaaaagagatatttttggattacttactcaagttggtcataattaacacacatcagtggaacttctgtactacaacgctggtgaaatttataaccacatgt

SEQ ID NO: 218 - DNA, 120 bp, Artificial Sequence
  Synthesized
attaacacacatcagtggaacttctgtactacaacgctggtgaaatttataaccacatgtttgacagcggaaaccctggaaaagcagctttcgacaaaagtcacaaaatgctaaggtgaa

SEQ ID NO: 219 - DNA, 120 bp, Artificial Sequence
  Synthesized
ttgacagcggaaaccctggaaaagcagctttcgacaaaagtcacaaaatgctaaggtgaaaaacgtttttcgtacctgcaaagtaaaaaatcacagagatttcaaaaactcacaagaaaa

SEQ ID NO: 220 - DNA, 120 bp, Artificial Sequence
  Synthesized
catttttgacatttcaaaaaaaaatgtaaagatacatacaaagttgtgtgttgtaagtggaacattctccaacacttccacatgcaattcttctccagtaagccaggaaatatcagtgtc

SEQ ID NO: 221 - DNA, 120 bp, Artificial Sequence
  Synthesized
aacattctccaacacttccacatgcaattcttctccagtaagccaggaaatatcagtgtcccaaccaattggtttcttctctctgaaaaatgtagacacaagcctttcttggttattaca

SEQ ID NO: 222 - DNA, 120 bp, Artificial Sequence
  Synthesized
tccatcgttagaaatttaaatggcaataaattaattttaagtttaccccaaataaatgtatcactcttggacaaacagcagggtaaagagatgtactcttttcttcaagtcagctccaga

SEQ ID NO: 223 - DNA, 120 bp, Artificial Sequence
  Synthesized
tcactcttggacaaacagcagggtaaagagatgtactcttttcttcaagtcagctccagaaaagacctacaacagcagaaaatggattgttcctggggtccaaaaaaaaaaaaaaaaaaa

SEQ ID NO: 224 - DNA, 120 bp, Artificial Sequence
  Synthesized
ctttttaaacaaaatttcacgtcacatacaaaccatacccatcctgaattctgtaaacagcacagcactctgggattagacctctcatcatcagtgctttctttagactgtctcggactg

SEQ ID NO: 225 - DNA, 120 bp, Artificial Sequence
  Synthesized
cacagcactctgggattagacctctcatcatcagtgctttctttagactgtctcggactgtaactccacaccttgcaggtacctatggtatcataaatatattgataagaggtaaaggga

SEQ ID NO: 226 - DNA, 120 bp, Artificial Sequence
  Synthesized
atattaaaactgactcaccactgtcctctgtttgttgggcaggaagactctaacgataggtttttgtggtgacttggggttgctccgtgccacatctgtgggattttgaaaaactgaaag

SEQ ID NO: 227 - DNA, 120 bp, Artificial Sequence
  Synthesized
tttttgtggtgacttggggttgctccgtgccacatctgtgggattttgaaaaactgaaagagatgaaggtagcactgaaaggctagaagaggaagaagatgtaacggtatccattgatgc

SEQ ID NO: 228 - DNA, 120 bp, Artificial Sequence
  Synthesized
agatgaaggtagcactgaaaggctagaagaggaagaagatgtaacggtatccattgatgcagagctagaaacagaaaaatcagttccgttccccagagattccaataactgttgttctct

SEQ ID NO: 229 - DNA, 120 bp, Artificial Sequence
  Synthesized
agagctagaaacagaaaaatcagttccgttccccagagattccaataactgttgttctctttgttggagtgcatctagcttgctggtgtattcttcataggcctataaaataaagcagac

SEQ ID NO: 230 - DNA, 120 bp, Artificial Sequence
  Synthesized
tcttttcaaaattactagatatgatactcaaaagcttacctccagatatattgatggtggattatgctccccaccaaatttgtccaatagggcctctatatgttcctgtgtcaacttaat

SEQ ID NO: 231 - DNA, 120 bp, Artificial Sequence
  Synthesized
attatgctccccaccaaatttgtccaatagggcctctatatgttcctgtgtcaacttaatcatttgtttgatattccacacctaaaaaatatttcaaaagaatttaaataaaaatcactt

SEQ ID NO: 232 - DNA, 120 bp, Artificial Sequence
  Synthesized
cagccgccgagcccggagtcgggagggcggcagggtggcgccagcactcacctcctccggaatggcagggtccgcagccgaagaggccgcggcgccggcgccggcgccggcctcgggctc

SEQ ID NO: 233 - DNA, 120 bp, Artificial Sequence
  Synthesized
aatggcagggtccgcagccgaagaggccgcggcgccggcgccggcgccggcctcgggctccatgtccccgttgaacagagcctggcccggctccgcgccgccaccaccgccaccgctcag

SEQ ID NO: 234 - DNA, 120 bp, Artificial Sequence
  Synthesized
catgtccccgttgaacagagcctggcccggctccgcgccgccaccaccgccaccgctcagcgccgccatcttataaccgagagccggggcccgagcggccgctgtcgggcggggaggggg

SEQ ID NO: 235 - DNA, 120 bp, Artificial Sequence
  Synthesized
agtatgccttaagaaaaaagtacaaattgtatttacataattacacactttgtctttgacttctttttcttctttttaccatctttgctcatcttttctttatgttttcgaatttctcga

SEQ ID NO: 236 - DNA, 120 bp, Artificial Sequence
  Synthesized
ttctttttcttctttttaccatctttgctcatcttttctttatgttttcgaatttctcgaactaatgtatagaaggcatcatcaacaccctgaaatacataaaaagtattaaaatgtgaa

SEQ ID NO: 237 - DNA, 120 bp, Artificial Sequence
  Synthesized
aattaatgtgctgaacttaaacttaccagattacattataatgcattttttaattttcacacagccaggagtcttttcttctttgctgatttttttcaatctgtattgtcggatctccct

SEQ ID NO: 238 - DNA, 120 bp, Artificial Sequence
  Synthesized
acagccaggagtcttttcttctttgctgatttttttcaatctgtattgtcggatctccctcaccaatgtataaaaagcatcctccactctctgcattgtaaaacacaacttctttaaagt

SEQ ID NO: 239 - DNA, 120 bp, Artificial Sequence
  Synthesized
ttgcagaaaacagatctgtatttatttcagtgttacttacctgtcttgtctttgctgatgtttcaataaaaggaattccataacttcttgctaagtcctgagcctgttttgtgtctactg

SEQ ID NO: 240 - DNA, 120 bp, Artificial Sequence
  Synthesized
tttcaataaaaggaattccataacttcttgctaagtcctgagcctgttttgtgtctactgttctagaaggcaaatcacatttatttcctactaggaccataggtacatcttcagagtcct

SEQ ID NO: 241 - DNA, 120 bp, Artificial Sequence
  Synthesized
ttctagaaggcaaatcacatttatttcctactaggaccataggtacatcttcagagtccttaactcttttaatttgttctctgggaaagaaaaaaaagttatagcacagtcattagtaac

SEQ ID NO: 242 - DNA, 120 bp, Artificial Sequence
  Synthesized
tgtcagcttattatattcaatttaaacccacctataatggtgaatatcttcaaatgatttagtattatttatggcaaatacacaaagaaagccctccccagtcctcatgtactggtccct

SEQ ID NO: 243 - DNA, 120 bp, Artificial Sequence
  Synthesized
agtattatttatggcaaatacacaaagaaagccctccccagtcctcatgtactggtccctcattgcactgtactcctcttgacctgctgtgtcgagaatatccaagagacaggtttctcc

SEQ ID NO: 244 - DNA, 120 bp, Artificial Sequence
  Synthesized
cattgcactgtactcctcttgacctgctgtgtcgagaatatccaagagacaggtttctccatcaattactacttgcttcctgtaggaatcctgagaagggagaaacacagtctggattat

SEQ ID NO: 245 - DNA, 120 bp, Artificial Sequence
  Synthesized
aaatgtcaataatgtattactcggggatttcctcttgaaattgtatgttttcttaatgtattaagtattgtaaggactttttacatactgcttaataacacctgtaggaaaagatgagaa

SEQ ID NO: 246 - DNA, 120 bp, Artificial Sequence
  Synthesized
ttaagtattgtaaggactttttacatactgcttaataacacctgtaggaaaagatgagaaacttttactcaattaatgctcaagtactttacgttactcttccgaaaggttatttaaatt

SEQ ID NO: 247 - DNA, 120 bp, Artificial Sequence
  Synthesized
tgcaccagtaatatgcatattaaaacaagatttacctctattgttggatcatattcgtccacaaaatgattctgaattagctgtatcgtcaaggcactcttgcctacgccaccagctcca

SEQ ID NO: 248 - DNA, 120 bp, Artificial Sequence
  Synthesized
acaaaatgattctgaattagctgtatcgtcaaggcactcttgcctacgccaccagctccaactaccacaagtttatattcagtcattttcagcaggccttataataaaaataatgaaaat

SEQ ID NO: 249 - DNA, 41 bp, Artificial Sequence
  Synthesized
ggattctaatacgactcactatagggatgcgacgtcgcagt

SEQ ID NO: 250 - DNA, 45 bp, Artificial Sequence
  Synthesized
ggattctaatacgactcactatagggtggactgcctggtccgaca